(19) United States
(12) Patent Application Publication
(10) Pub. No.: US 2012/0258971 A1
(43) Pub. Date: Oct. 11, 2012

(54) THERAPEUTIC AGENT FOR CHRONIC PAIN

(75) Inventors: Shin-Ichi Niwa, Fukushima City (JP); Shinichi Konno, Fukushima City (JP); Satoshi Kasahara, Fukushima City (JP); Hirobumi Mashiko, Fukushima City (JP); Koji Otani, Fukushima City (JP)

(73) Assignee: OTSUKA PHARMACEUTICAL CO., LTD., Chiyoda-ku, Tokyo (JP)

(21) Appl. No.: 13/395,364

(22) PCT Filed: Feb. 26, 2010
(86) PCT No.: PCT/JP2010/053032

§ 371 Date: Jun. 28, 2012

(30) Foreign Application Priority Data
Sep. 11, 2009 (JP) 2009-211021

(51) Int. Cl. A61K 31/496; A61P 29/00; C07D 295/12

(52) U.S. Cl. 514/253.07; 544/363

(57) ABSTRACT

This invention provides a novel therapeutic agent for chronic pain. The therapeutic agent for chronic pain comprises aripiprazole as an active ingredient.

### TECHNICAL FIELD

The present invention relates to a therapeutic agent for chronic pain comprising aripiprazole as an active ingredient.

### BACKGROUND ART

Chronic pain refers to severe and distressing pain that may interfere with daily life and that continues for six months or more. This type of pain is called “persistent somatoform pain disorder” by the International Statistical Classification of Diseases and Related Health Problems, 10th Revision (ICD-10). It is suggested that psychological factors have a major impact on the onset and exacerbation of chronic pain; however, its cause remains unknown.

Among chronic pain patients who see an orthopedic surgeon, more than a few have inconsistent neurological signs, suffer from intractable pain, and presumably have psychiatric problems in their backgrounds. Some patients have a bitter experience in which physicians do not properly assess their psychiatric problems, but simply repeat invasive treatments, thereby causing the further exacerbation of pain.

Currently, various drugs are used in an attempt to relieve chronic pain; however, they are not always satisfactory in terms of their analgesic effect. Accordingly, effective therapeutic agents for chronic pain are in demand.

Meanwhile, aripiprazole is a useful atypical antipsychotic drug for the treatment of schizophrenia (e.g., PTL 1 and PTL 2).

### CITATION LIST

### Patent Literature

PTL 1: U.S. Pat. No. 4,734,416

PTL 2: U.S. Pat. No. 5,006,528

### SUMMARY OF INVENTION

### Technical Problem

An object of the present invention is to provide a novel therapeutic agent for chronic pain.

### Solution to Problem

The present inventors conducted extensive research to achieve the above object. As a result, when aripiprazole was administered to a chronic pain patient, significant analgesic effects were observed. Thus, the inventors found that aripiprazole is effective as a therapeutic agent for chronic pain. The present invention was accomplished upon further studies based on this finding.

More specifically, the present invention provides a therapeutic agent for chronic pain comprising aripiprazole as an active ingredient.

Item 1. A therapeutic agent for chronic pain comprising aripiprazole as an active ingredient.

Item 2. The therapeutic agent for chronic pain according to Item 1, which comprises aripiprazole or an acid addition salt or solvate thereof as an active ingredient.

Item 3. The therapeutic agent for chronic pain according to Item 1 or 2, further comprising a pharmaceutically acceptable carrier.

Item 4. Use of aripiprazole for the production of a therapeutic agent for chronic pain.

Item 5. Aripiprazole for use in the treatment of chronic pain.

Item 6. A method for treating chronic pain, comprising administering an effective amount of aripiprazole to a patient.

Item 7. The method according to Item 6, wherein the aripiprazole is administered to a patient in a dose of about 0.05 to 10 mg per kg of body weight per day.

### Advantageous Effects of Invention

The therapeutic agent for chronic pain of the present invention comprises aripiprazole as an active ingredient, and exhibits a significant analgesic effect.

### BRIEF DESCRIPTION OF DRAWING

FIG. 1 is a graph showing a course of treatment in which aripiprazole and other drugs were continuously administered to a chronic pain patient.

### DESCRIPTION OF EMBODIMENTS

The present invention is a therapeutic agent for chronic pain comprising aripiprazole as an active ingredient.

Aripiprazole is a compound having the chemical name of 7-{4-[4-(2,3-dichlorophenyl)-1-piperazinyl]butoxy}-3,4-dihydrocarbostyril or 7-{4-[4-(2,3-dichlorophenyl)-1-piperazinyl]butoxy}-3,4-dihydro-2(1H)-quinolinone.

Aripiprazole may be not only in the free form but also in the form of an acid addition salt with a pharmaceutically acceptable acid. Examples of such acids include inorganic acids, such as sulfuric acid, nitric acid, hydrochloric acid, phosphoric acid, and hydrobromic acid; and organic acids, such as acetic acid, p-toluenesulfonic acid, methanesulfonic acid, oxalic acid, maleic acid, fumaric acid, malic acid, tartaric acid, citric acid, succinic acid, and benzoic acid. As with aripiprazole in a free form, the acid addition salts can also be used as active ingredient compounds in the present invention.

Moreover, aripiprazole may be in the form of a solvate (e.g., a hydrate or a solvate with an alcohol).

The above free, acid addition salt, and solvate forms of aripiprazole may include crystalline and/or amorphous forms. The crystalline forms include various crystal polymorphs.

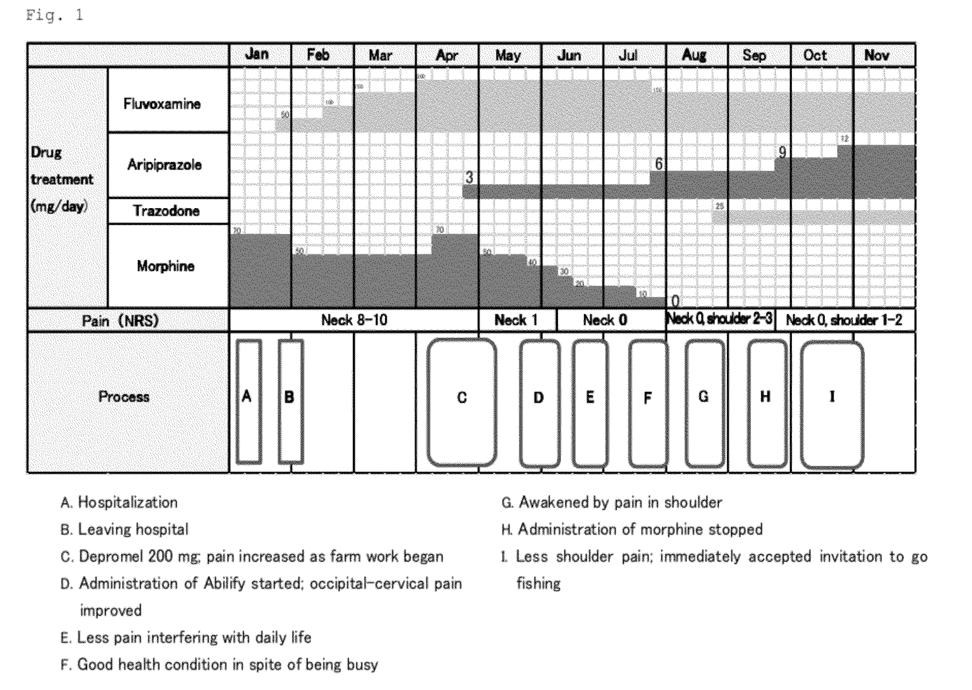

Aripiprazole exhibits significant analgesic activity for patients with chronic pain diseases (including fibromyalgia, etc., which are systemic chronic pain disorders) to improve their symptoms. Therefore, aripiprazole is highly useful as a therapeutic agent for chronic pain. Specifically, for example, as shown in Example 1 and FIG. 1, symptoms of a chronic pain patient were not improved by the administration of an analgesic (morphine) and an antidepressant (fluvoxamine); however, the symptoms were markedly improved by the administration of aripiprazole.

The chronic pain therapeutic agent of the present invention may further comprise a pharmaceutically acceptable carrier in the above forms of aripiprazole. Examples of pharmaceutically acceptable carriers include diluents and excipients, such as fillers, extenders, binders, humectants, disintegrators, surfactants, and lubricants, which are generally used in pharmaceutical preparations. The chronic pain therapeutic agent of the present invention may be used in the form of a general pharmaceutical preparation. Examples of the form include tablets, flash-melt tablets, pills, powders, solutions, suspensions, emulsions, granules, capsules, suppositories, injections (e.g., solutions and suspensions), troches, nasal sprays, transdermal patches, etc.

The route of administration of the chronic pain therapeutic agent of the present invention is not particularly limited, and the therapeutic agent is administered by a route suitable to the form of the therapeutic agent, the patient's age, the patient's sex, and other conditions (e.g., severity of the disease). For example, tablets, pills, solutions, suspensions, emulsions, granules, and capsules are administered orally. Injections are intravenously administered singly or mixed with typical fluid replacements, such as glucose solutions or amino acid solutions, or singly administered intramuscularly, intracutaneously, subcutaneously, or intraperitoneally. Suppositories are administered intrarectally.

The dosage of the chronic pain therapeutic agent of the invention is suitably selected according to the method of use, the patient's age, the patient's sex, and other conditions, and the severity of the disease. Generally, the amount of aripiprazole is about 0.05 to 10 mg per kg of body weight per day. Furthermore, the preparation in a dosage unit form can contain aripiprazole in an amount of about 1 to 100 mg, and preferably 1 to 30 mg, per unit dose.

All documents cited herein are incorporated by reference.

### EXAMPLES

The present invention is described in detail below using an Example; however, the present invention is not limited thereto.

### Example

Morphine, fluvoxamine, aripiprazole, and other drugs were administered for about 11 months to a patient who was diagnosed as a chronic pain sufferer with chronic occipital-cervical pain that had continued for ten years or more. The intensity of the pain in the patients' occipital-cervical region (neck) was evaluated over time. FIG. 1 shows the course of treatment.

The intensity of pain was evaluated using a numerical rating scale (NRS) in which pain was orally reported on an 11-step scale (0 to 10). This evaluation method numerically expresses (quantifies) the degree of pain on a scale of 0 (no pain) to 10 (worst conceivable pain). This method provides a good reflection of the degree of pain of a patient before and after treatment.

Referring to FIG. 1, first, morphine hydrochloride tablets (produced by Dainippon Sumitomo Pharma Co., Ltd.) were orally administered in a dose of 70 mg/day to a chronic pain patient (body weight: 55 kg); however, the NRS value in the neck was as high as 8 to 10, and the pain was not relieved. From the 4th week of the first month, in addition to morphine, oral administration of fluvoxamine (Depromel tablets; produced by Meiji Seika Pharma Co., Ltd.) in a dose of 50 mg/day was started. Although the dose of fluvoxamine was gradually increased, the NRS value was still as high as 8 to 10, and the pain was not relieved at all.

Then, from the 4th week of the 4th month, aripiprazole (Abilify tablets; produced by Otsuka Pharmaceutical Co., Ltd.) was orally administered in a dose of 3 mg/day. As a result, the NRS value was dramatically reduced to 1 in the first week of the 5th month. From the second week of the 6th month, the NRS value was 0, indicating that there was no neck pain. Furthermore, even when the administration of morphine was stopped from the 8th month, the NRS value remained at 0. These results confirmed that morphine and fluvoxamine could not relieve the pain of the chronic pain patient, while aripiprazole could dramatically reduce the pain.

Thereafter, when the dose of aripiprazole (Abilify tablets; produced by Otsuka Pharmaceutical Co., Ltd.) was increased to 9 mg/day after the 4th week of the 9th month, and further increased to 12 mg/day after the 4th week of the 10th month, the NRS value was 0, and no change was observed.

The above results demonstrate that aripiprazole is very effective as a therapeutic agent for chronic pain.

1. A therapeutic agent for chronic pain comprising aripiprazole as an active ingredient.

2. The therapeutic agent for chronic pain according to claim 1, which comprises aripiprazole or an acid addition salt or solvate thereof as an active ingredient.

3. The therapeutic agent for chronic pain according to claim 1, further comprising a pharmaceutically acceptable carrier.

4. Use of aripiprazole for the production of a therapeutic agent for chronic pain.

5. Aripiprazole for use in the treatment of chronic pain.

6. A method for treating chronic pain, comprising administering an effective amount of aripiprazole to a patient.

7. The method according to claim 6, wherein the aripiprazole is administered to a patient in a dose of about 0.05 to 10 mg per kg of body weight per day.

8. The therapeutic agent for chronic pain according to claim 2, further comprising a pharmaceutically acceptable carrier.
